(19) United States
(12) Patent Application Publication
(10) Pub. No.: US 2024/0315678 A1
(43) Pub. Date: Sep. 26, 2024

(54) CONNECTION ARRANGEMENT FOR A SURGICAL INSTRUMENT OR IN A SURGICAL INSTRUMENT

(71) Applicant: KARL STORZ SE & Co. KG, Tuttlingen (DE)

(72) Inventors: Lisa DIETERLE, Tuttlingen (DE); Sven GRÜNER, Tuttlingen (DE)

(21) Appl. No.: 18/610,752

(22) Filed: Mar. 20, 2024

(30) Foreign Application Priority Data
Mar. 21, 2023 (DE) 10 2023 107 109.2

(51) Int. Cl. A61B 17/00

(52) U.S. Cl. CPC A61B 17/00; A61B 2017/00477

(57) ABSTRACT

A connection arrangement for a surgical instrument or in a surgical instrument, includes an instrument base which is designed for controlling and/or operating a high-frequency tool of the surgical instrument, and a connection body of a high-frequency connector. The connection body is designed for predetermined orientation in a connection axis of the high-frequency connector within the instrument base. The instrument base forms a receptacle for the connection body and the receptacle has an orientation portion. The connection body has a corresponding orientation portion. The connection body, by insertion into the receptacle by the orientation portions, can orient itself at a predetermined angle of rotation on the connection axis. The invention further relates to a surgical instrument having a connection arrangement.

### FIELD OF THE INVENTION

The present invention relates to a connection arrangement for a surgical instrument or in a surgical instrument. The invention further relates to a surgical instrument with a connection arrangement.

### TECHNICAL BACKGROUND

Surgical instruments are used for different applications. For example, they can be designed as microinvasive medical instruments and comprise a manipulation device at a proximal end, a long and generally thin shaft extending from the proximal end to a distal end of the instrument, and a tool or other effecting device for gripping, crushing, coagulating, cutting, punching or other uses at the distal end of the instrument. In the shaft, one or more transmission devices for transmitting a force and/or a torque extend from the manipulation device at the proximal end to the effecting device at the distal end. Thus, in microinvasive medical instruments with an electrosurgical function, especially in bipolar electrosurgical microinvasive medical instruments, the transmission device is often also involved in the transmission of electrical power from the proximal end to the distal end.

Advantageously, the instruments can be disassembled into individual components, so that different tools, different shafts and other connection elements can be combined. Advantageously, a system can therefore be made available having different and therefore versatile applications.

High-quality microinvasive medical instruments are also generally reusable. To simplify cleaning after use and to permit replacement of a defective component and/or an alternative use of different components, a high-quality microinvasive medical instrument is advantageously able to be disassembled.

Firstly, in the case of a medical instrument which is able to be disassembled and in which the transmission device is also involved in the transmission of electrical power, a mechanically separable and safely recoverable electrical contact to the transmission device must be able to be produced in particular at the proximal end of the instrument.

Furthermore, only components that can also be used in a functional manner should be combinable with each other. The aim is thus to ensure that, in the case of a plurality of different systems, for example, a surgical instrument with monopolar, bipolar, or currentless operation, only those components that belong to the same system can be combined with one another. This can prevent incorrect use, damage, and the like.

Thus, the document DE 10 2017 124 775 A1 discloses a microinvasive medical instrument having an instrument base and an accessory shaft which can be inserted into the instrument base and which has a transmission device for transmitting electrical power and for transmitting a force and/or a torque from a proximal position to a distal position. A contact device is arranged in the instrument base and, at the side facing away from the accessory shaft, is connected to a plug-in contact.

A disadvantage of such instruments is that the plug-in contact, for example, a high-frequency connector, is permanently mounted in the instrument base, i.e. in the handle element.

Therefore, removable high-frequency connectors are also available that can be inserted as a plug-in element into the instrument base and form a connection arrangement. However, assembly is often complicated, since an exact orientation is required.

### SUMMARY OF THE INVENTION

Against this background, the object of the present invention is to provide an improved connection arrangement.

According to the invention, this object is achieved by a connection arrangement having the features of claim 1 and by a surgical instrument having the features of claim 17.

Accordingly, the following is provided:
A connection arrangement for a surgical instrument or in a surgical instrument, with an instrument base which is designed for controlling and/or operating a high-frequency tool of the surgical instrument, with a connection body of a high-frequency connector, wherein the connection body is designed for predetermined orientation in a connection axis of the high-frequency connector within the instrument base, wherein the instrument base forms a receptacle for the connection body and the receptacle has an orientation portion, wherein the connection body has a corresponding orientation portion, and wherein the connection body, by insertion into the receptacle by the orientation portions, can orient itself at a predetermined angle of rotation on the connection axis.
A surgical instrument comprising such a connection arrangement, wherein the surgical instrument is operated as a bipolar or monopolar instrument.

The finding on which the present invention is based is that a rotary fixing of the high-frequency connector in the receptacle of the instrument base is advantageous if the said connector is to be mounted unambiguously and without further intervention.

The concept on which the present invention is based is to allow the high-frequency connector to be inserted into the receptacle at a predetermined angle of rotation on a connection axis of the high-frequency connector, by means of orientation portions arranged on the receptacle and on the high-frequency connector, and in a self-aligning manner.

In other words, a substantially round high-frequency connector in the form of a plug can be fixed with a defined position of rotation in the receptacle, which is formed as a kind of bore or blind hole, in such a way that assembly is possible without further orientation measures and/or without repeated testing.

Correctly aligned insertion during assembly is not essential to ensure the operation of the surgical instrument, since the orientation portions provide automatic rotational orientation during the insertion. Therefore, the high-frequency connector does not have to be oriented in the precisely correct position when being inserted into the instrument base. In this way, complex guide geometries between the high-frequency connector and the instrument base or complex orientation tools for assembly are avoided.

Furthermore, it is advantageously possible in this way to form a means of preventing the rotation of the high-frequency connector relative to the receptacle. It is thus possible to dispense with pinning, gluing or any other form-fit or cohesively bonded connection between the high-frequency connector and the receptacle, which cuts down on the number of work steps or assembly steps and avoids errors of orientation.

The high-frequency connector can have different configurations. In particular, it is in the form of a plug-in element and has a substantially round cross section. The cross section can taper conically, which also permits optimization of production by an injection molding process.

As an instrument base, a handle element can be provided to which the further components of the surgical instrument can be attached or mounted. Alternatively or in addition, for robotic surgical applications, the instrument base itself can be a robot interface or an instrument base that can be mounted with a robot coupling.

Advantageously, the surgical instrument is operated as a bipolar instrument. In a further embodiment, the surgical instrument can be a monopolar instrument.

A connection body of the high-frequency connector can be understood as a main body of the high-frequency connector on which all other components of the high-frequency connector, in particular the poles and contacts, are attached or premounted or premountable.

An orientation portion can be understood as a component geometry that permits an orientation on the connection axis and/or fixes the rotation of the two components, i.e. of the high-frequency connector and the instrument base, in an angle of rotation about the connection axis.

A predetermined angle of rotation on the connection axis is to be understood as an orientation at a desired angle of rotation in the mounted state, in which the high-frequency connector is correctly oriented when fully inserted or pushed into the receptacle. The orientation portions allow the high-frequency connector to self-align to this predetermined angle of rotation during insertion. In particular, this can also prevent damage that the high-frequency connector, for example, the contacts and the instrument base, could suffer as a result of incorrect orientation at the time of insertion.

If the high-frequency connector is mounted in the instrument base, i.e. in a handle for example, it is particularly important that a rotational position of the high-frequency connector corresponds to a setpoint position, so that internal contacts and/or passages interact smoothly with the instrument base, and with an instrument shaft that can be inserted therein, and contact them correctly.

Advantageous embodiments and developments will become clear from the further subclaims and also from the description given with reference to the figures in the drawing.

According to an advantageous embodiment, the orientation portions can be designed as a projection and as an indent corresponding to the latter. Advantageously, a form fit between the high-frequency connector and the instrument base can thus be produced in which an already existing geometry can be utilized. For example, as an orientation portion, it is possible to use an already existing groove which is formed in the high-frequency connector, in particular in the connection body, for example, in order to accommodate other components. Furthermore, a groove can be used which is formed in the high-frequency connector, for example, for production-related reasons. Furthermore, a groove may already be provided in the receptacle of the instrument base, which groove can now be used as an orientation portion. The same applies, for example, to a lug or bulge which is already formed on the connection body or in the receptacle for production-related reasons or for other reasons. Advantageously, no dedicated geometry and no additional components are thus required to prevent rotation, and instead, the already existing features are used. The groove can be interpreted here as an indent, and the lug or bulge as a projection.

According to a further development, the projection can protrude from an inner surface of the receptacle. This allows the projection to be at a desired position and/or to extend over a certain length of the receptacle. If an indent is formed on the connection body corresponding to the projection, a rotation of the high-frequency connector relative to the instrument base is advantageously prevented.

According to one embodiment, the projection can be oriented along the connection axis and have an elongate shape. This advantageously permits an oriented insertion of the connection body into the receptacle, since even at an early stage there is contact between the two orientation portions.

According to an advantageous embodiment, the projection can extend over at least half a length of the receptacle, the length being defined in the direction of the connection axis. The projection can be placed in contact with an insertion opening of the receptacle so that, already upon contact of the connection body with the receptacle, the orientation at the predetermined angle of rotation on the connection axis is as it were self-aligning. It is thus possible to visually perceive the angle at which the high-frequency connector has to be inserted into the receptacle.

According to a preferred embodiment, the projection can be arranged spaced apart from an insertion opening of the receptacle, such that the projection likewise forms a stop which limits insertion of the connection body into the receptacle on the connection axis. The projection can be rounded at the end oriented in the direction of the insertion opening, so that a simple “threading in” can take place upon contact with the indent in the connection body. The two orientation portions can therefore slide against each other.

Advantageously, the projection can be designed as a negative form of the indent. This can prevent a relative shift between the high-frequency connector and the instrument base, since the orientation portions, i.e. in particular the projection and the indent, can produce a kind of form fit. In particular, twisting from the predetermined angle of rotation in the installed state can be prevented.

According to a particularly preferred embodiment, the projection for the orientation of the connection body in the receptacle can have side edges conically tapering toward the insertion opening. Advantageously, a shape can thus be formed which can be produced by an injection molding process and furthermore has advantages during assembly. At the time of assembly, a conical design, i.e. a tapering in the direction of the insertion opening, affords the advantages that the components, i.e. the connection body and the receptacle, can be easily “found”. When the connection body is then pushed further into the receptacle, the increasingly widening projection, in particular in the form of a kind of widening rib, can orient the connection body to a final position in which the connection body is intended to remain free of play.

According to an advantageous embodiment, the connection body can have a conical design and taper in the direction of a plug-in connector. This results, for example, in advantages in production by an injection molding process. For example, the connection body can be made of an insulating plastic. On the connection body, as a kind of main body, at least two electrical poles can be arranged. One of the poles can be formed as a contact plate extending from a plug-in connector, at one end of the connection body, to an opposite end of the connection body. Contact tabs, in particular four contact tabs, can be formed at the opposite end. For example, the contact plate can extend along an orientation portion. In this case, a shaping within the connection body, designed to receive the contact plate, can also serve as an orientation portion. As a result, an already existing geometry of the connection body can be used as an orientation portion. A second pole can likewise be guided from one end to an opposite end on the connection body and runs in particular within the connection body.

According to a further development, the connection body can have at least one oblique or curved insertion contour to allow insertion of the orientation portions when the connection body is oriented at an angle other than the predetermined angle of rotation on the connection axis.

The insertion contour is arranged in particular at the opposite end of the connection body, which is pushed first into the receptacle. In other words at the end where the contact tabs are provided, not the plug-in connector. The insertion contour forms in particular a curved edge region of the connection body, which region is inclined with respect to the connection axis and in particular protects the contact tabs from damage or bending. The insertion contour is therefore preferably not formed along a cross section through the connection body that runs transversely, in particular at right angles, to the connection axis. Rather, the insertion contour is formed as an oblique contact surface, which is oriented obliquely with respect to a right-angled cross-sectional area of the connection body.

According to one embodiment, the indent can form a receptacle for a contact plate of a first pole, the contact plate having a thickness less than a depth of the indent. This can, for example, ensure that a sufficiently large groove remains that can serve as an orientation portion. In particular, the indent can be formed over the full length of the connection body, while the projection has a shorter length.

According to one embodiment, in an end region adjacent to the insertion contour, the indent can transition continuously into the insertion contour. Advantageously, contacting of the connection body with the receptacle is possible in such a way that, upon further insertion of the connection body into the receptacle, an orientation at the predetermined angle of rotation can take place at an early stage. When the orientation portion of the connection body contacts the insertion contour, the orientation portion of the connection body is guided in the direction of the orientation portion of the receptacle, thereby permitting correct insertion, or “threading in” of the two orientation portions.

In an advantageous embodiment, the connection body can have recesses which serve as a grip and/or identification for the rotational orientation in the predetermined angle of rotation of the connection body relative to the receptacle. For example, the connection body can be held in the hand by an operator at the recesses. When this person grips the recesses, a correct orientation at the predetermined angle of rotation takes place “automatically”, for example since the recesses are oriented symmetrically to a longitudinal axis of the surgical instrument, and therefore also to a longitudinal axis of the instrument base. The recesses are preferably arranged in the region that runs in the direction of a plug-in connector on the connection body. In particular, the recesses are therefore not in contact with the insertion contour and are preferably formed at a distance from the insertion contour. This allows the connection body to be inserted into the receptacle in a manner not influenced by the recesses.

According to an advantageous embodiment, the connection body can be at least partially cylindrical. For example, it can have a circular cylindrical design. Thus, a kind of plug-in element can be formed which has a substantially round cross section. It is advantageous if the connection body is at least partially cylindrical in the front region, i.e. in the region which is guided first into the receptacle. In a further embodiment, the connection body can taper in the direction of the plug-in connector.

According to a further development, the recesses can form part of the tapering and have a cylindrical surface on an opposite side of the connection body. Thus, the insertion contour can be formed in a manner uninfluenced by the tapering, and at the same time ergonomic handling of the high-frequency connector in the form of a plug can be made possible.

According to an advantageous embodiment, the insertion contour can intersect the cylindrical surface. While the cylindrical surface preferably extends rotationally symmetrically about the connection axis, the insertion contour can be oriented obliquely on the connection axis such that it does not extend in a cross section at right angles to the connection side. In this way, a part of the cylindrical surface is removed which can, for example, form insertion bevels, in order to guide a projection in the receptacle into the indent in the connection body.

According to an advantageous embodiment, the connection body can be designed as a uniaxially demoldable injection molded part. It is advantageous that all surfaces converge conically in the direction of an axis, so that simple demolding is possible. Advantageously, there are also no sharp edges present on the connection body, and therefore only round surfaces are formed. This also affords advantages during demolding after the injection molding process.

According to a further development, the instrument base can be formed as a connecting device, for example to a manual guide element executed as a handle, to a manipulator coupling, or to a robot receptacle.

The above embodiments and developments can be combined with one another in any desired manner, provided this is feasible. Further possible embodiments, developments and implementations of the invention also comprise combinations, not explicitly mentioned, of features of the invention described hereinabove or hereinbelow with regard to the exemplary embodiments. In particular, a person skilled in the art will also add on individual aspects as improvements or additions to the respective basic form of the present invention.

### STATEMENT ON THE CONTENT OF THE DRAWING

The present invention is explained in more detail below on the basis of the exemplary embodiments given in the schematic figures of the drawing. In the drawing:

FIG. 1 shows a longitudinal section through a surgical instrument;

FIG. 2 shows an instrument base;

FIG. 3 shows a receptacle in an instrument base;

FIG. 4 shows a cross section through the receptacle from FIG. 3;

FIG. 5 shows a longitudinal section through the receptacle from FIG. 3;

FIG. 6 shows a connection body;

FIG. 7 shows a longitudinal section through the connection body from FIG. 6;

FIG. 8 shows a further view of a connection body;

FIG. 9 shows a further view of a connection body;

FIG. 10 shows a further view of a connection body;

FIG. 11 shows a further view of a connection body with plug-in connector;

FIG. 12 shows a further view of a connection body from FIG. 11;

FIG. 13 shows a further view of a connection body; and

FIG. 14 shows a further view of the connection body from FIG. 13.

The appended figures of the drawing are intended to impart further understanding of the embodiments of the invention. They illustrate embodiments and, in association with the description, serve to explain principles and concepts of the invention. Other embodiments and many of the advantages mentioned will be apparent from the drawings. The elements of the drawings are not necessarily shown true to scale with respect to one another.

In the figures of the drawing, elements, features and components that are the same, have the same function and the same effect are, unless stated otherwise, each given the same reference signs.

### DESCRIPTION OF EXEMPLARY EMBODIMENTS

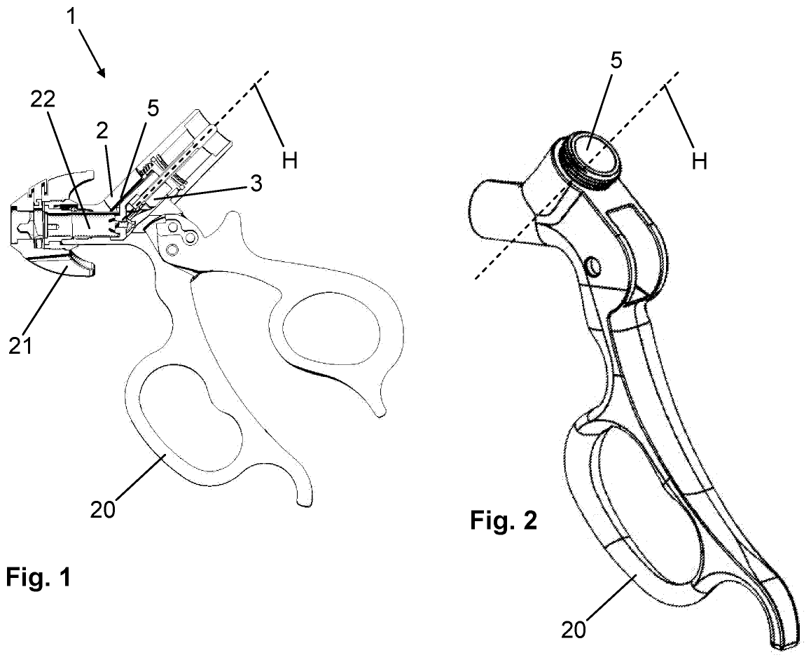

FIG. 1 shows a longitudinal section through a surgical instrument. Advantageously, the surgical instrument is operated as a bipolar instrument. An embodiment operated as a monopolar instrument is also conceivable. The surgical instrument has an instrument base 2 having a receptacle 5 in which a connection body 3 is received. The receptacle 5 leads within the instrument base 2 to a shaft opening 22, in which there is arranged a receiving element, with a rotary wheel arrangement 21 arranged therein. Furthermore, a manipulation device 20 with a thumb ring is provided.

FIG. 2 shows an instrument base 2 in an isometric representation. It will be seen that the receptacle 5 is substantially cylindrical and forms an opening along a connection axis H. The connection axis H extends in particular on a plane which is spanned by a longitudinal section through the surgical instrument, as is shown in FIG. 1. Furthermore, the connection axis H can run at any desired angle on this plane.

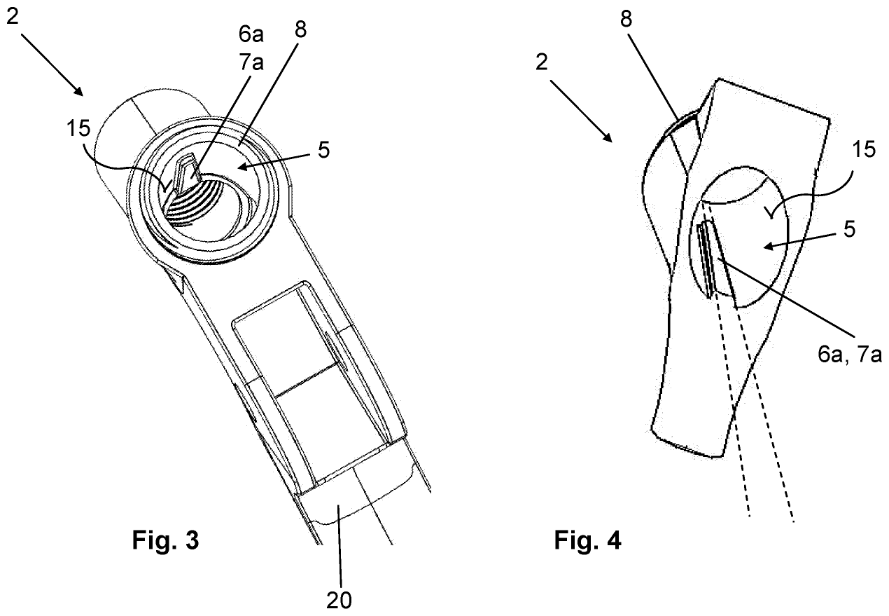

FIG. 3 shows a receptacle 5 in an instrument base 2. An orientation portion 6a, designed as a projection 7a, is provided in the receptacle 5. The orientation portion 6a is formed on an inner surface 15 of the receptacle 5. The orientation portion 6a therefore forms a kind of stop, against which the connection body 3 (not shown) can support itself. In the embodiment shown, the orientation portion 6a is arranged spaced apart from an insertion opening 8. Furthermore, the orientation portion 6a extends along the connection axis H. In a further embodiment not shown, the orientation portion 6a can have a different geometry and, for example, can adjoin the insertion opening 8. Furthermore, a plurality of such orientation portions 6a can be provided on the inner surface 15.

FIG. 4 shows a cross section through the receptacle from FIG. 3. The cross section runs through the instrument base 2 and through the orientation portion 6a. It will be seen that the two side edges of the orientation portion 6a, or of the projection 7a, run conically toward each other. Preferably, the side edges are shaped conically in the direction of the insertion opening 8, as is shown in FIG. 4. The illustrated tapering of the projection 7a in the direction of the insertion opening 8 is advantageous in several respects. For example, if the component is produced by an injection molding process, a core has to be used that must be pulled out from the finished injection molded part during demolding. Parallel surfaces lying opposite each other create unnecessary friction and are therefore to be avoided for design reasons. The conical flanks can therefore advantageously form draft angles. Furthermore, an advantage is afforded during assembly, since the two parts, i.e. the connection body 3 and the receptacle 5 with the orientation portions 6a, 6b arranged thereon, are very easily “found” when joining them. If the connection body 3 is pushed in further, the increasingly widening projection 7a can orient the connection body 3 to a final position in which the connection body 3 remains free of play in the receptacle 5.

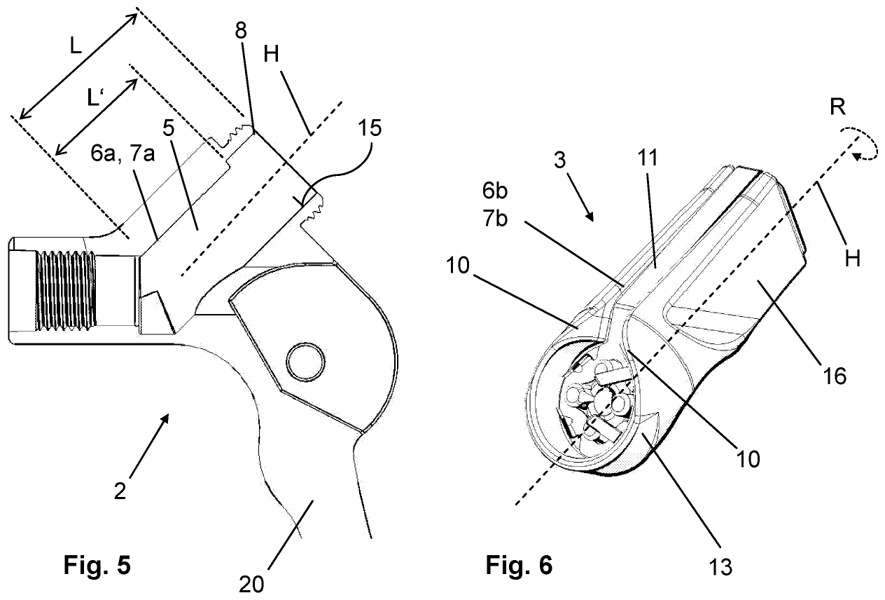

FIG. 5 shows a longitudinal section through the receptacle from FIG. 3. It will be seen that the length L′ of the orientation portion 6a is shorter than the length L of the complete receptacle 5. In the embodiment shown, the orientation portion 6a, designed as a projection 7a, extends over at least half of the length L of the receptacle 5. It is likewise conceivable that the orientation portion 6a is arranged at a different position on the inner surface 15.

FIG. 6 shows a connection body 3. The connection body 3 can be inserted, as shown, into the receptacle 5 according to FIG. 5. For this purpose, the connection body 3 has an orientation portion 6b, which is designed as an indent 7b. The indent 7b is likewise oriented along the connection axis H. Furthermore, a contact surface 11 of a first pole 12 extends in the indent 7b. The indent 7b therefore forms a receptacle for the contact plate 11, which contact plate 11 has a thickness less than a depth of the indent 7b. Thus, the orientation portion 6b can be formed in an already existing geometry on the connection body 3. Furthermore, an insertion contour 10 is provided on the connection body 3 and transitions continuously into the orientation portion 6b. The insertion contour 10 serves for inserting the orientation portion 6b when the connection body 3 is oriented at an angle other than the predetermined angle of rotation R on the connection axis H. The insertion contour 10 is therefore designed as an oblique or curved surface, which intersects a cylindrical surface 13 on the connection body 3. Such a geometry can simplify the insertion of the connection body 3 into the receptacle 5.

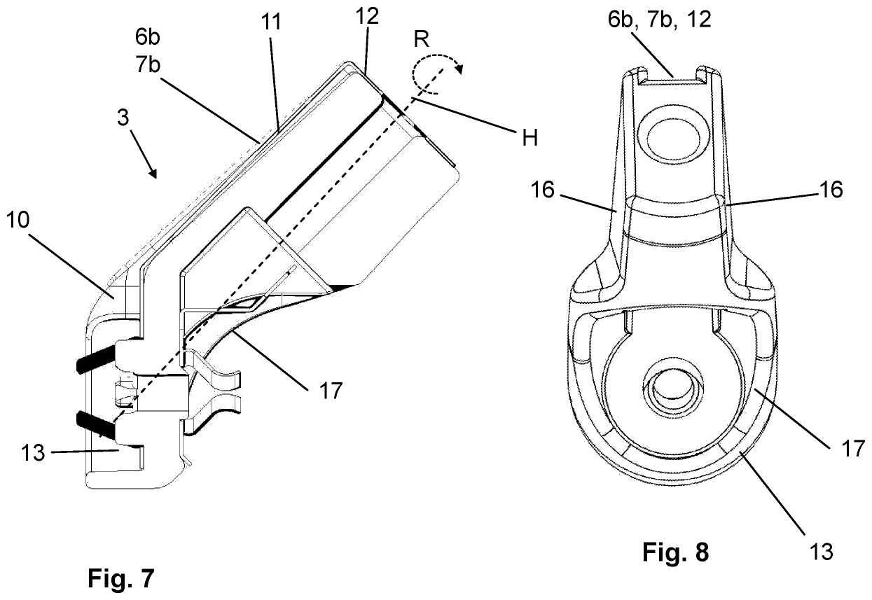

FIG. 7 shows a longitudinal section through the connection body 3 from FIG. 6. It will be seen that the contact plate 11 extends over the length of the connection body 3 and, at one end, forms a first pole 12, which is formed at the opposite end with contact tabs. The contact tabs are protected by the cylindrical surface 13 and the insertion contour 10. Furthermore, an indent 17 is provided on the connection body 3, by means of which material can be saved, and a cross section is formed that can be easily produced by an injection molding process and easily demolded.

FIG. 8 shows a further view of a connection body 3. It will be seen that the connection body 3 tapers conically, wherein recesses 16 are provided at an end region 2, and the cylindrical surface 13 is arranged at the opposite end region. The recesses 16 can form part of the tapering. Furthermore, the recesses 16 can serve as a handle and/or identification for the rotational orientation in the predetermined angle of rotation R. Thus, it is possible to simply visually identify at which position the orientation portion 6b has to be aligned with the first pole 12 in order to be inserted symmetrically into the receptacle 5.

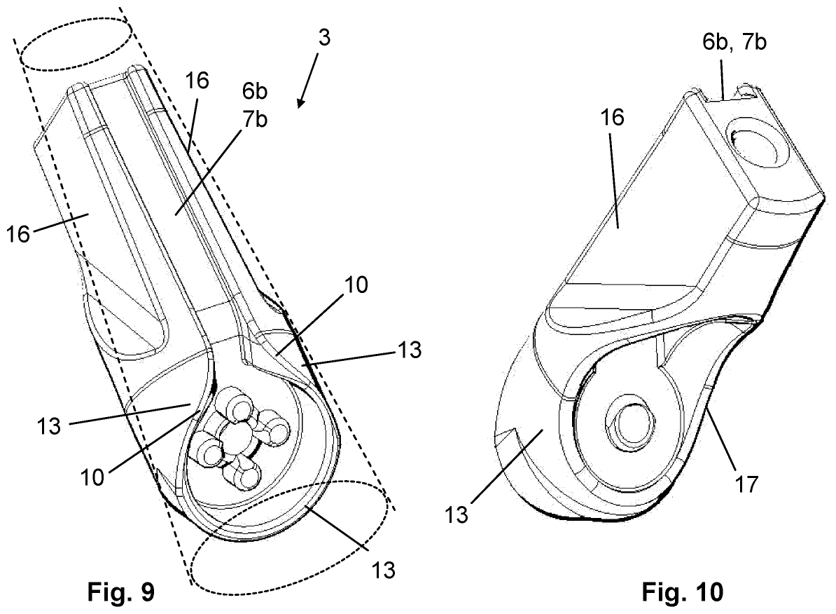

FIG. 9 shows a further view of a connection body 3, the conical shape being indicated by dashed lines. A shape of this kind is particularly suitable for an injection molding process, since it only has round surfaces, which can be advantageously demolded.

FIG. 10 shows a further view of a connection body 3.

This view reveals the indents 17 on the rear of the connection body 3, by which the connection body 3 can be designed in a way that saves material.

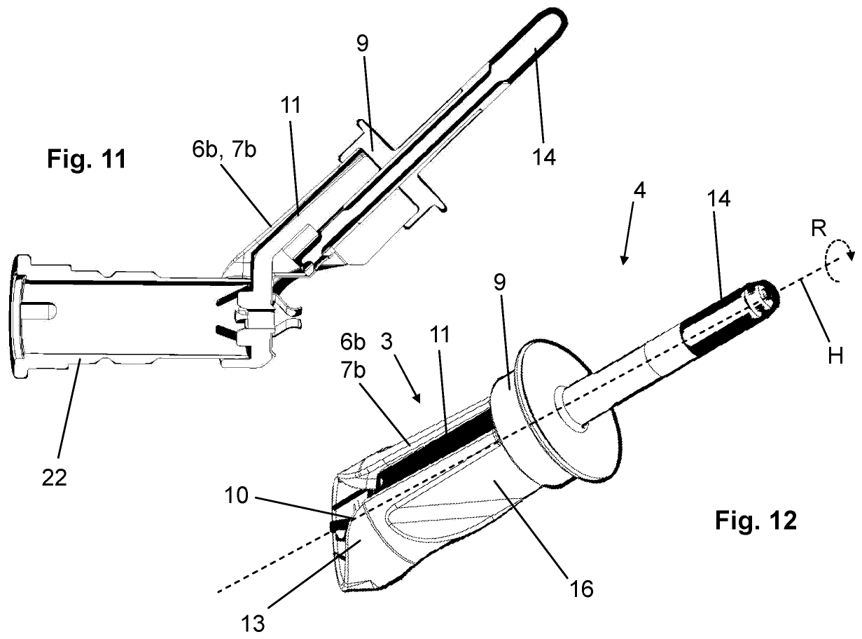

FIG. 11 shows a further view of a connection body 3 with plug-in connector 9. Within the plug-in connector 9, a second pole 14 is provided which is likewise guided to the opposite end on the connection body 3. At the opposite end, the connection body 3 can be connected to a receiving element in the shaft opening 22, shown in FIG. 1.

As is shown in FIG. 12, the plug-in connector 9 can be connected on the side to the connection body 3, on which the recesses 16 are provided. The illustration shows a type of plug-in element which can be designed as a high-frequency connector for a bipolar instrument handle. The connection body 3 can form a kind of main body and is made in particular of an insulating plastic. One of the two poles, in particular the first pole 12, is transmitted via the contact plate 11 from the plug-in connector 9 to the instrument shaft and there contacted by four contact tabs. The second pole 14 runs in the interior of the plug-in connector element.

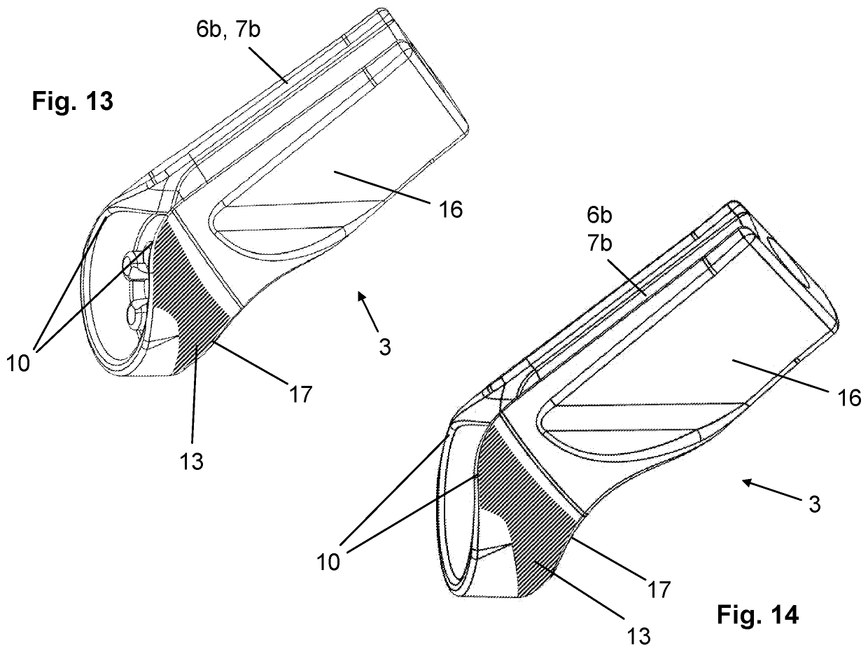

FIG. 13 and FIG. 14 show further views of a connection body 3, wherein the cylindrical surface 13 is indicated as a dashed surface. The dashed surface illustrated can be used to precisely align and simplify insertion of the connection body 3 into the receptacle 5. Snagging during insertion can be ruled out. It will also be seen that the insertion contour 10 intersects the cylindrical surface 13, thereby as it were removing a part of the cylindrical surface. This part serves to simplify orientation of the two orientation portions 6a and 6b relative to each other.

Although the present invention has been described above in its entirety on the basis of preferred exemplary embodiments, it is not limited to these, and instead it can be modified in a variety of ways. Thus, for example, the geometry of the connection body 3 can be formed differently, as long as the described function of the orientation by the orientation portions 6a, 6b is ensured.

### LIST OF REFERENCE SIGNS

1 connection arrangement
2 instrument base
3 connection body
4 high-frequency connector
5 receptacle
6a orientation portion
6b orientation portion
7a projection
7b indent
8 insertion opening
9 plug-in connector
10 insertion contour
11 contact plate
12 first pole
13 cylindrical surface
14 second pole
15 inner surface
16 recess
17 indent
20 manipulation device
21 rotary wheel arrangement
22 shaft opening
H connection axis
L, L′ length
R angle of rotation

What is claimed is:

1. A connection arrangement for a surgical instrument or in a surgical instrument, the connection arrangement comprising:
an instrument base which is configured to control and/or operate a high-frequency tool of the surgical instrument; and
a connection body of a high-frequency connector, wherein the connection body is configured for predetermined orientation in a connection axis of the high-frequency connector within the instrument base,
wherein the instrument base forms a receptacle for the connection body and the receptacle has an orientation portion, wherein the connection body has a corresponding orientation portion, and wherein the connection body, by insertion into the receptacle by the orientation portions, can orient itself at a predetermined angle of rotation on the connection axis.

2. The connection arrangement as claimed in claim 1, wherein the orientation portions are configured as a projection and as an indent, the projection corresponding to the indent.

3. The connection arrangement as claimed in claim 2, wherein the projection protrudes from an inner surface of the receptacle.

4. The connection arrangement as claimed in claim 2, wherein the projection is oriented along the connection axis and has an elongate shape.

5. The connection arrangement as claimed in claim 2, wherein the projection extends over at least half a length of the receptacle, the length being defined in a direction of the connection axis.

6. The connection arrangement as claimed in claim 2, wherein the projection is arranged spaced apart from an insertion opening of the receptacle.

7. The connection arrangement as claimed in claim 2, wherein the projection, for the orientation of the connection body in the receptacle, has side edges tapering conically to the insertion opening.

8. The connection arrangement as claimed in claim 1, wherein the connection body is conical and tapers in a direction of a plug-in connector.

9. The connection arrangement as claimed in claim 1, wherein the connection body has at least one oblique or curved insertion contour to allow insertion of the orientation portions when the connection body is oriented at an angle other than the predetermined angle of rotation on the connection axis.

10. The connection arrangement as claimed in claim 2, wherein the indent forms a receptacle for a contact plate of a first pole, the contact plate having a thickness less than a depth of the indent.

11. The connection arrangement as claimed in claim 9, wherein the orientation portions are configured as a projection and as an indent, the projection corresponding to the indent and wherein, in an end region adjacent to the insertion contour, the indent transitions continuously into the insertion contour.

12. The connection arrangement as claimed in claim 1, wherein the connection body has recesses which serve as a grip and/or identification for the rotational orientation at the predetermined angle of rotation of the connection body relative to the receptacle.

13. The connection arrangement as claimed in claim 1, wherein the connection body is at least partially cylindrical.

14. The connection arrangement as claimed in claim 12, wherein the connection body is conical and tapers in a direction of a plug-in connector and wherein the recesses form part of the taper and, on an opposite side, the connection body has a cylindrical surface.

15. The connection arrangement as claimed in claim 13, wherein the connection body is conical and tapers in a direction of a plug-in connector and wherein the recesses form part of the taper and, on an opposite side, the connection body has a cylindrical surface.

16. The connection arrangement as claimed in claim 13, wherein the connection body has at least one oblique or curved insertion contour to allow insertion of the orientation portions when the connection body is oriented at an angle other than the predetermined angle of rotation on the connection axis and wherein the insertion contour intersects a cylindrical surface of the at least partially cylindrical connection body.

17. The connection arrangement as claimed in claim 14, wherein the connection body has at least one oblique or curved insertion contour to allow insertion of the orientation portions when the connection body is oriented at an angle other than the predetermined angle of rotation on the connection axis and wherein the insertion contour intersects the cylindrical surface.

18. The connection arrangement as claimed in claim 1, wherein the connection body is configured as a uniaxially demoldable injection molded part.

19. A surgical instrument comprising a connection arrangement, the connection arrangement comprising:
an instrument base which is configured to control and/or operate a high-frequency tool of the surgical instrument; and
a connection body of a high-frequency connector, wherein the connection body is configured for predetermined orientation in a connection axis of the high-frequency connector within the instrument base,
wherein the instrument base forms a receptacle for the connection body and the receptacle has an orientation portion, wherein the connection body has a corresponding orientation portion, and wherein the connection body, by insertion into the receptacle by the orientation portions, can orient itself at a predetermined angle of rotation on the connection axis, and wherein the surgical instrument is operated as a bipolar or monopolar instrument.

20. The surgical instrument as claimed in claim 19, wherein the orientation portions are configured as a projection and as an indent, the projection corresponding to the indent.
